(19) United States
(12) Patent Application Publication
(10) Pub. No.: US 2025/0248928 A1
(43) Pub. Date: Aug. 7, 2025

(54) NANOPARTICLES AND NANOPARTICLE-RELEASING VAGINAL RINGS

(71) Applicant: Yale University, New Haven, CT (US)

(72) Inventors: W. Mark Saltzman, New Haven, CT (US); Elias Quijano, New Haven, CT (US); Yang Deng, New Haven, CT (US); Molly Grun, New Haven, CT (US); Bernadette Marquez-Nostra, New Haven, CT (US)

(21) Appl. No.: 18/856,787

(22) PCT Filed: Apr. 13, 2023
(86) PCT No.: PCT/US2023/018528

§ 371 Date: Oct. 14, 2024

Related U.S. Application Data
(60) Provisional application No. 63/331,025, filed on Apr. 14, 2022.

(51) Int. Cl. A61K 9/00

(52) U.S. Cl. CPC A61K 9/0036

(57) ABSTRACT

Nanoparticles and nanoparticle-releasing vaginal rings for intermediate- to long-term delivery of drugs to the female genital and reproductive tract have been developed. The nanoparticles are loaded with drugs and coated with a sheddable poly(ethylene glycol) (PEG) layer that promotes mucus penetration and then converts to an adhesive form after penetration. This platform technology readily distributes drug through the mucosa and throughout the vaginal tissue, enhances local retention of drugs within the vaginal tissue, thereby providing a sustained delivery of drugs beyond the natural shedding and turnover of vaginal mucous and epithelial cells.

### CROSS-REFERENCE TO RELATED APPLICATIONS

This application claims the benefit of and priority to U.S. Ser. No. 63/331,025, filed on Apr. 14, 2022, which is specifically incorporated by reference herein in their entirety.

### STATEMENT REGARDING FEDERALLY SPONSORED RESEARCH

This invention was made with government support under Grant No. R01 EB000487 awarded by the National Institutes of Health. The government has certain rights in the invention.

### BACKGROUND OF THE INVENTION

In 1970, U.S. Pat. No. 3,545,439 assigned to the UpJohn Company was published describing “an improved resilient annular device for intravaginal placement and retention . . . containing an effective amount of a medicament which is capable of passage through the drug-permeable polymeric material”. This first description of the concept of a drug-releasing vaginal ring heralded an intense period of activity in ring development through the 1970s and 1980s, mainly focused around contraception and hormone replacement therapy and involving various ring designs. The World Health Organization (WHO) was at the forefront of this research with their program for the development of a silicone elastomer steroidal contraceptive ring to help contain the burgeoning population growth.

Vaginal rings are flexible, torus-shaped, silicone elastomer or thermoplastic devices that provide long-term, sustained or controlled delivery of pharmaceutical substances to the vagina for either local or systemic effect. Designed to be readily inserted and removed by the woman herself, they are generally positioned in the upper third of the vagina adjacent to the cervix. Although the exact location of ring placement is generally not critical for clinical efficacy, it may have implications for comfort in some women.

Intravaginal drug delivery devices, including IVRs, are typically formed from biocompatible polymers and contain a drug released by diffusion through the polymer matrix. The devices may be inserted into the vaginal cavity and the drug absorbed by the surrounding body fluid through the vaginal tissue. In some IVR designs, the drug is uniformly dispersed or dissolved throughout the polymer matrix (monolithic system). In other designs, the drug is confined to an inner core within the ring (reservoir system). Monolithic systems are expected to show Fickian diffusion-controlled drug release whereby the release rate decreases with time. Reservoir systems may exhibit a zero order release of loaded drugs. The silicone IVR first made in circa 1970 was designed to elute hormones for a 30-day duration and provide sustained drug levels in the range of 10 to 100 μg/day. Since then, there has been little innovation in IVR technology.

Seven vaginal ring products have been marketed, five of which are fabricated from silicone elastomers (ESTRING®, FEMRING®, PROGERING®, FERTIRING® AND ANNOVERA®) and two from thermoplastic polymers (NUVARING® and ORNIBEL®). An antiretroviral-releasing silicone elastomer vaginal ring for HIV prevention is used Europe but was withdrawn in the US due to poor efficacy. See Malcolm et al. Int J Womens Health. 2012; 4: 595-605.

The active pharmaceutical ingredients in these vaginal ring products are all highly potent, small molecular weight (<540 g/mol), lipophilic (log P>2) steroids or antiviral molecules that can readily permeate the hydrophobic silicone elastomers and thermoplastic polymers to offer clinically significant release rates. Current IVR technology is inadequate to meet the high topical dose requirements of antiviral drug delivery as demonstrated by the withdrawal of the anti-HIV ring as of March 2022. Delivery from silicone (for example, formed of a biomedical grade polymer such as SILASTIC MDX4-4210 silicone elastomer base (Dow Corning, Thailand) as a base material) and ethylene vinyl acetate-based IVR), show only in vitro and in vivo daily delivery rates in microgram quantities, rather than milligram quantities, as is required for delivery of antivirals such as human immunodeficiency virus (HIV) prophylaxis. The drug release from silicone and ethylene vinyl acetate polymers is therapeutically insignificant, primarily due to the hydrophilicity of drugs such as tenovir and resultant low solubility in the elastomeric polymers commonly used for IVRs, although higher levels were reported using IVRs made of hydrophilic aliphatic polyether urethanes (HPU) (Lubrizol Advanced Materials (Wickliffe, OH).

It is therefore an object of the present invention to provide a composition and method to provide controlled and sustained drug delivery to the vagina, over a prolonged period of time, including delivery of drugs requiring higher loading than currently available devices can provide.

It is a further object to provide a composition and method which is economical, reproducible, and controllable, as well as acceptable to the recipient.

### SUMMARY OF THE INVENTION

Nanoparticles (NP) and nanoparticle-releasing intravaginal rings (IVR) for intermediate- to long-term delivery of drugs to the female genital and reproductive tract have been developed. This platform technology readily distributes drug throughout the vaginal tissue, enhances local retention of drugs, and provides a sustained delivery of drugs beyond the natural shedding and turnover of vaginal mucous and epithelial cells. The NP nanoparticles are formed of a biodegradable hydrophobic polymer loaded with drug. They are coated with a sheddable poly(ethylene glycol) (PEG) layer, typically in the form of PEO dendrimer or hyperbranched polyglycerol that promotes mucus penetration by the NPs, which is then cleaved off within the tissue to make the nanoparticles tissue adhesive. The NPs can be incorporated into IVRs that slowly release the NPs for longer duration of drug action. These are well suited for vaginal delivery of hormonal, anti-infective and therapeutic agents, with the goal of improving the residence time and distribution throughout the vaginal and ectocervical tissues and can be controlled by the woman. Use of reservoirs allow for high loading of drug, as well as longer term release than currently available IVRs.

NP formulations are based on a polymer such as the biodegradable FDA approved polyhydroxy ester, poly(lactic acid) (PLA), poly(amine-co-ester)s, and hyperbranched polyglycerols (HPG) conjugated to form polymer. PLA-HPG NPs were oxidized to aldehyde terminated PLA-HPG_{ALD} NPs and conjugated to hydrazide terminated PEG (NH₂NH-PEG) by a reversible Schiff base bond. HPG has many vicinal diol groups, which enable the attachment of multiple ligands on the HPG corona. These vicinal diols are easily oxidized to aldehyde groups that allow attachment of many kinds of ligands under mild conditions. PEG coated PLA-HPG NP and PLA-HPG NPs have comparable penetration through mucus, but PEG coated PLA NPs have a half-life of six hours compared to 10 hours for PLA-HPG. The PEG coating is shed in the presence of low pH (<5.0) or in the presence of large concentrations of competing proteins. After the PEG coating is shed, an aldehyde rich surface is exposed, allowing for interaction and binding to proteins to the vaginal epithelium.

To extend the duration of the drug-loaded NPs in the vagina, the NPs are placed into individual millimeter scale reservoirs within an otherwise solid IVR made of a polymer such as poly(ethylene-co-vinyl acetate) (EVAc). The NPs are released within minutes in water in the absence of caps. Degradable “caps” are used to seal each reservoir and are made from polymers that degrade at different rates, either due to selection of specific molecular weights (MW) and/or the thickness of the cap. In a preferred embodiment, degradation time for a cap formed from a degradable polymer such as PLGA or PLA film of specific MW and composition is used to control release of the NPs from the IVR. These NPs, which can be easily loaded with antivirals such as ELVITEGRAVIR® (Gilead) and CMX157 (a lipophilic prodrug of tenofovir), will significantly extend the duration of action of these agents, potentially to one week after a single intravaginal dose. Further, these can be used for the sustained release of NPs loaded with drugs, extending the duration of action to at least one month after a single administration.

Examples show that HPG-PLA NPs, (mean diameter 86 nm) provide unprecedented residence time after vaginal delivery. These NPs are incorporated into IVRs that slowly release the NPs for longer duration of activity. In one embodiment, the NPs are loaded into release reservoirs in the IVR, each reservoir is sealed with a degradable polymer cap, and each polymer cap is designed to degrade at a different time after placement of the IVR. Sheddable PEG NPs can be synthesized from degradable materials that are known to be safe in humans, formulated for penetration of the mucus barrier, bundled into a solid form that can be loaded into reservoirs in the IVR, and readily rehydrated upon exposure to water as free, non-aggregated NPs.

By releasing NPs that are loaded with drug instead of free drug molecules, the IVR benefits from the many advantages of NPs for drug delivery to the reproductive mucosa, including improved pharmacokinetics/bioavailability. The performance of the NP/IVR combination can be tuned over a wide range, providing features that are unavailable in any other formulation.

Studies conducted in a non-human primate model demonstrate that the bioadhesive nanoparticles (“BNPs”) were useful for drug and gene delivery to the vagina. BNPs radiolabelled using a long-lasting radiotracer (⁸⁹Zr, t_{1/2}=3.3 d), allowed tracking of nanoparticle retention with excellent sensitivity over many days. The results showed that BNPs were retained in the vaginal canal for at least 5 days and did not enter the uterus or systemic circulation. By contrast, a small molecule (⁸⁹Zr-DFO), was detected in systemic circulation just 60 min after vaginal administration. Multiple vaginal doses of BNPs did not increase the expression of inflammatory biomarkers in vaginal fluid or plasma, indicating that the nanoparticles are biocompatible.

### BRIEF DESCRIPTION OF THE DRAWINGS

FIGS. 1A-1C are a schematic of the particle surface chemistry, showing conversion from a mucus-penetrating stealth coating to a bioadhesive coating, as a function of the sheddable PEG surface (FIG. 1A) on particles of PLA-HPG. The diol groups on PLA-HPG are oxidized by NaIO₄ to aldehyde groups (FIG. 1B) then conjugated to Hydrazide-PEG to yield sheddable PEG surface (FIG. 1C). FIG. 1B is a graph of the size distribution (10 to 70 nm) of the particles and FIG. 1C is a graph of controlled release of a drug (camptothecin, CPT) from PLA-HPG NPs.

FIG. 2A is a cross-sectional view and FIG. 2B is a side view of the vaginal ring device including 15 reservoirs that are loaded with nanoparticles.

FIGS. 3A-3E shows method for producing sealed reservoirs that will release the nanoparticles after dissolution of a degradable polymer cap.

FIG. 4 is a graph of the diffusion of mucus-penetrating NPs in human cervical mucus. NPs that contained high densities of low molecular weight PEG units (density increases are recorded on the x-axis) diffused as fast in mucus as in water. MW of PEG (2K—circles, 5K—squares, and 10K—triangles) were tested for their affect on the rate of diffusion.

FIG. 5A is a micrograph of the particles and FIG. 5B is a graph of the size distribution (% versus size in nm) of the particles.

FIG. 6 shows the predicted drug release of NPs from the ring (percent) over time (hours), when the caps are designed to degrade over different time periods, between 2 and 30 days.

FIG. 7 is a graph of cumulative nanoparticle release over time, from 2 to 30 hours.

FIG. 8A is a graph showing in vitro release of ⁸⁹Zr from ⁸⁹Zr-BNPs in simulated vaginal fluid at pH 5, 6, and 7. FIG. 8B is a graph of ⁸⁹Zr-BNP retention and distribution after vaginal application, showing organ-specific activity for 5 days post-vaginal administration. FIG. 8C is a table of decay-corrected activity in the vaginal canal over 5 days.

FIGS. 9A and 9B are graphs of the changes in cytokine concentration in (9A) vaginal fluid and (9B) plasma 48 hr after vaginal BNP administration, normalized to baseline expression for each animal. Cytokines that were below the detection limit are identified under the x=0 line (BDL).

### DETAILED DESCRIPTION OF THE INVENTION

### I. Definitions

Intravaginal rings (“IVR”) are small plastic devices that provide continuous release of hormones, typically over 21 days, that are inserted into the vagina of a woman who wants to prevent pregnancy.

Nanoparticle (“NP”), as used herein, generally refers to a particle of any shape having a diameter from about 1 nm up to, but not including, about 1 micron, more typically from about 5 nm to about 500 nm. The preferred size range of the mucus penetrating NPs is preferably from about 5 nm to about 100 nm. Nanoparticles having a spherical shape are generally referred to as “nanospheres”.

Mean particle size, as used herein, generally refers to the statistical mean particle size (diameter) of the particles in a population of particles. The diameter of an essentially spherical particle may be referred to as the physical or hydrodynamic diameter. The diameter of a non-spherical particle may refer preferentially to the hydrodynamic diameter. As used herein, the diameter of a non-spherical particle refers to the largest linear distance between two points on the surface of the particle. Mean particle size can be measured using methods known in the art, such as dynamic light scattering.

Monodisperse and homogeneous size distribution are used interchangeably herein and describe a plurality of nanoparticles or microparticles where the particles have the same or nearly the same diameter or aerodynamic diameter. As used herein, a monodisperse distribution refers to particle distributions in which at least 80, 85, 90, 95%, or an integer therebetween, or greater of the distribution lies within 5% of the mass median diameter or aerodynamic diameter.

Hydrophilic, as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

Lipophilic refers to compounds having an affinity for lipids.

Amphiphilic refers to a molecule combining hydrophilic and lipophilic (hydrophobic) properties.

Hydrophobic as used herein refers to substances that lack an affinity for water; tending to repel and not absorb water as well as not dissolve in or mix with water.

As used herein, an amphiphilic polymer is one which has one end formed of a hydrophilic polymer and one end formed of a hydrophobic polymer. As a result, when dispersed into a mixture of water and low water-solubility solvent such as many of the organic solvents, the hydrophilic end orients into the water and the hydrophobic end orients into the low water-solubility end.

Self-assembling refers to the use of amphiphilic polymers, alone or in mixture with hydrophilic and/or hydrophobic polymers, which orient in a mixture of aqueous and non-aqueous solvents to form particles, wherein the hydrophilic ends orient with the other hydrophilic ends and the hydrophobic ends orient with the other hydrophobic ends.

The term stealth refers to the ability of a nanoparticle to evade immune recognition to enhance its circulation time in vivo, and thereby its chances of reaching the target.

The term surfactant as used herein refers to an agent that lowers the surface tension of a liquid.

The term active agent refers to a therapeutic, prophylactic or diagnostic agent that can be administered to prevent or treat one or more symptoms of a disease or disorder. Prophylactic agents are used to prevent a disease or disorder. These agents can be nucleic acids, small molecules (defined herein as having a molecular weight of 1500 daltons or less), proteins, peptides, or peptidomimetic, carbohydrates or sugars, lipid, or a combination thereof.

Pharmaceutically acceptable, as used herein, refers to compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration.

Biocompatible and biologically compatible, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory or immune response when administered to a patient.

The term biodegradable, as used herein, generally refers to a material that degrades or erodes under physiologic conditions to smaller units or chemical species that are capable of being metabolized, eliminated, or excreted by the subject. The degradation time is a function of composition and morphology. Degradation times can be from hours to years.

The term implant, as used herein, generally refers to a device that is inserted into the body.

Molecular weight, as used herein, generally refers to the relative average chain length of the bulk polymer, unless otherwise specified. In practice, molecular weight can be estimated or characterized using various methods including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

### II. Nanoparticles and IVRs

The compositions include an intravaginal ring (IVR) that contains and releases nanoparticles (NP) that release hormone over an extended period of time following placement in the vagina, in an amount effective to prevent pregnancy. Release periods may be the same as currently available IVRs used to prevent pregnancy (i.e., releasing over a 21 day period), or may provide longer term release over a period of months. In alternative embodiments, the nanoparticles can be formulated in a gel or suppository that can be directly placed in the vagina to release hormone over an extended period of time. In some embodiments, the nanoparticles can be formulated in a gel or suppository, which can then be loaded into an IVR for placement in the vagina.

### A. Nanoparticles

Nanoparticle-based therapeutics and diagnostics offer immense potential in a wide range of biomedical applications. Drug-loaded nanoparticles have garnered great interest in drug delivery as a strategy to improve the pharmacokinetics of drugs (Banik, et al. WIREs Nanomed Nanobiotechnol 2016, 8, 271-299) However, the impact of nanoparticle-based drug delivery in clinical medicine has been limited, with only a small number of FDA-approved formulations to date. (Anselmo, et al. Bioeng. Transl. Med. 2016, 1, 10-29; An Update. Bioeng. Transl. Med. 2019, 4, 1-16) Most formulations tested in clinical trials ultimately fail to show beneficial effects due to their rapid blood clearance and low drug delivery efficiency (Cho et al. Biomaterials 2012, 33, 1190-1200; Qian, et al. Polym. Chem. 2016, 7, 3300-3310).

A widely used method to enhance the blood circulation time of nanoparticles is based on coating the surface with a dense layer of poly(ethylene glycol) (PEG), which reduces opsonization and subsequent clearance by the mononuclear phagocytosis system (Rabanel, et al. ACS Appl. Mater. Interfaces 2015, 7, 10374-10385). While this “stealthy” PEG-coating effectively inhibits the adsorption of serum proteins on the nanoparticle surface via steric repulsion, the same characteristic inherently reduces their uptake into cells (Cao, et al. ACS Nano 2020, 14, 3563-3575). Several physicochemical parameters of PEG-coated nanoparticles such as size (Walkey, et al. J. Am. Chem. Soc. 2012, 134, 2139-2147), shape (Kinnear, et al. Chem. Rev. 2017, 117, 11476-11521), rigidity (Hui, et al. ACS Nano 2019, 13, 7410-7424; Mullner, et al. ACS Nano 2015, 9, 1294-1304), and surface functionality (Sutton, et al. Pharm. Res. 2007, 24, 1029-1046); Deng, et al Proc. Natl. Acad. Sci. U.S.A 2016, 113, 11453-11458) have been investigated as methods to modulate cell uptake and protein adsorption.

Nanoparticles are used for drug and gene delivery throughout the vaginal tissue, but current formulations do not persist for long in the vagina due to mucosal shedding and persistent mucus secretion. PEG coatings may increase particle penetration beyond the mucous barrier, but prevents adherence to the vaginal epithelium, which is essential for long term release. Therefore, an NP system with a sheddable PEG layer was created to facilitate mucosal diffusion, which upon reaching the vaginal epithelium, is shed, presenting an adhesive surface on the NP. The adhesive surface is then free to attach to the vaginal epithelium, substantially increasing intravaginal NP retention. Because of the potential for epithelial shedding, these NPs were incorporated into an IVR that can provide multiple doses of fresh NPs with a sheddable PEG coating.

The NP formulations are based on a core polymer composed of a biodegradable, biocompatible hydrophobic polymer such as poly(lactic acid) (PLA) in combination with an outer shell formed of hyperbranched polyglycerols (HPG), which may be covalently bound to the hydrophobic polymer to form the HPG outer coating and inner hydrophobic polymer core or be presented as branched or dendrimeric polyalkylene glycols forming the outside shell of the NPs. HPG having exposed hydroxyl groups facilitates movement through mucosa and into tissue, where enzymes cleave the linkage of the hydroxyls to the HPG, to expose tissue adhesive groups such as aldehydes and amines.

In a preferred embodiment, the hydrophobic core forming polymer is biodegradable. Exemplary biodegradable polymers include polymers that are insoluble or sparingly soluble in water that are converted chemically or enzymatically in the body into water-soluble materials. Biodegradable polymers can include soluble polymers crosslinked by hydrolyzable cross-linking groups to render the crosslinked polymer insoluble or sparingly soluble in water. Preferred hydrophobic polymers include polyhydroxy esters such as polylactic acid (PLA), polyglycolic acid (PGA), copolymers there (PLGA), polyanhydrides, polyhydroxyalkanoates, and polyamine-co-esters, all biodegradable and approved by the US Food and Drug Administration for administration (FDA), to a human or animal.

In preferred embodiments, the active agents are encapsulated within and/or complexed with a polyhydroxy acid ester such as poly(lactic-co-glycolic acid) (PLGA), poly(lactic acid) (PLA), or poly(glycolic acid) (PGA), to form a particle with nanometer dimensions. Poly(lactic-co-glycolic acid) (PLGA), or (PLG), is a copolymer which is used in many FDA approved therapeutic devices, owing to its biodegradability and biocompatibility. In some embodiments, the particle includes the biodegradable polymer blended with, or covalently bound to one or more additional polymers.

The hydrophobic core polymers are associated with, most preferably covalently bound to, HPG. For example, HPG can be covalently coupled to a polymer having carboxylic acid groups, such as PLA, PGA, or PLGA using DIC/DMAP.

Hyperbranched polyglycerol is a highly branched polyol containing a polyether scaffold. Hyperbranched polyglycerol can be prepared using techniques known in the art. It can be formed from controlled etherification of glycerol via cationic or anionic ring opening multibranching polymerization of glycidol. For example, an initiator having multiple reactive sites is reacted with glycidol in the presence of a base to form hyperbranched polyglycerol (HPG). Suitable initiators include, but are not limited to, polyols, e.g., triols, tetraols, pentaols, or greater and polyamines, e.g., triamines, tetraamines, pentaamines, etc. In one embodiment, the initiator is 1,1,1-trihydroxymethyl propane (THP).

A formula for hyperbranched polyglycerol as described in EP 2754684 is

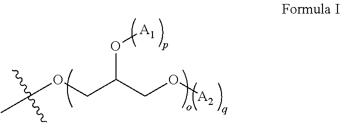

wherein o, p and q are independently integers from 1-100,
wherein A₁ and A₂ are independently

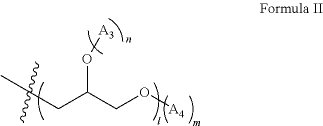

wherein l, m and n are independently integers from 1-100.
wherein A₃ and A₄ are defined as A₁ and A₂, with the proviso that A₃ and A₄ are hydrogen, n and m are each 1 for terminal residues.

The surface properties of the HPG can be tuned based on the chemistry of vicinal diols. For example, the surface properties can be tuned to provide stealth particles, i.e., particles that are not cleared by the MPS due to the presence of the hydroxyl groups; adhesive (sticky) particles, i.e., particles that adhere to the surface of tissues, for example, due to the presence of one or more reactive functional groups, such as aldehydes, amines, oxime, or O-substituted oxime that can be prepared from the vicinal hydroxyl moieties and/or targeting by the introduction of one or more targeting moieties which can be conjugated directly or indirectly to the vicinal hydroxyl moieties. Indirectly refers to transformation of the hydroxy groups to reactive functional groups that can react with functional groups on molecules to be attached to the surface, such as active agents and/or targeting moieties, etc.

The hyperbranched nature of the polyglycerol allows for a much higher density of hydroxyl groups, reactive functional groups, and/or targeting moieties than polyethylene glycol. For example, the particles described herein can have a density of surface functionality (e.g., hydroxyl groups, reactive functional groups, and/or targeting moieties) of at least about 1, 2, 3, 4, 5, 6, 7, or 8 groups/nm².

The molecular weight of the HPG can vary. For example, in those embodiments wherein the HPG is covalently attached to the materials or polymers that form the core, the molecular weight can vary depending on the molecular weight and/or hydrophobicity of the core materials. The molecular weight of the HPG is generally from about 1,000 to about 1,000,000 Daltons, from about 1,000 to about 500,000 Daltons, from about 1,000 to about 250,000 Daltons, or from about 1,000 to about 100,000 Daltons. In those embodiments wherein the HPG is covalently bound to the core materials, the weight percent of HPG of the copolymer is from about 1% to about 50%, such as about 1, 2, 3, 4, 5, 10, 15, 20, 25, 30, 35, 40, 45 or 50%.

In some embodiments, the HPG is covalently coupled to a hydrophobic material or a more hydrophobic material, such as a polymer. Upon self-assembly, particles are formed containing a core containing the hydrophobic material and a shell or coating of HPG. HPG coupled to the polymer PLA is shown below:

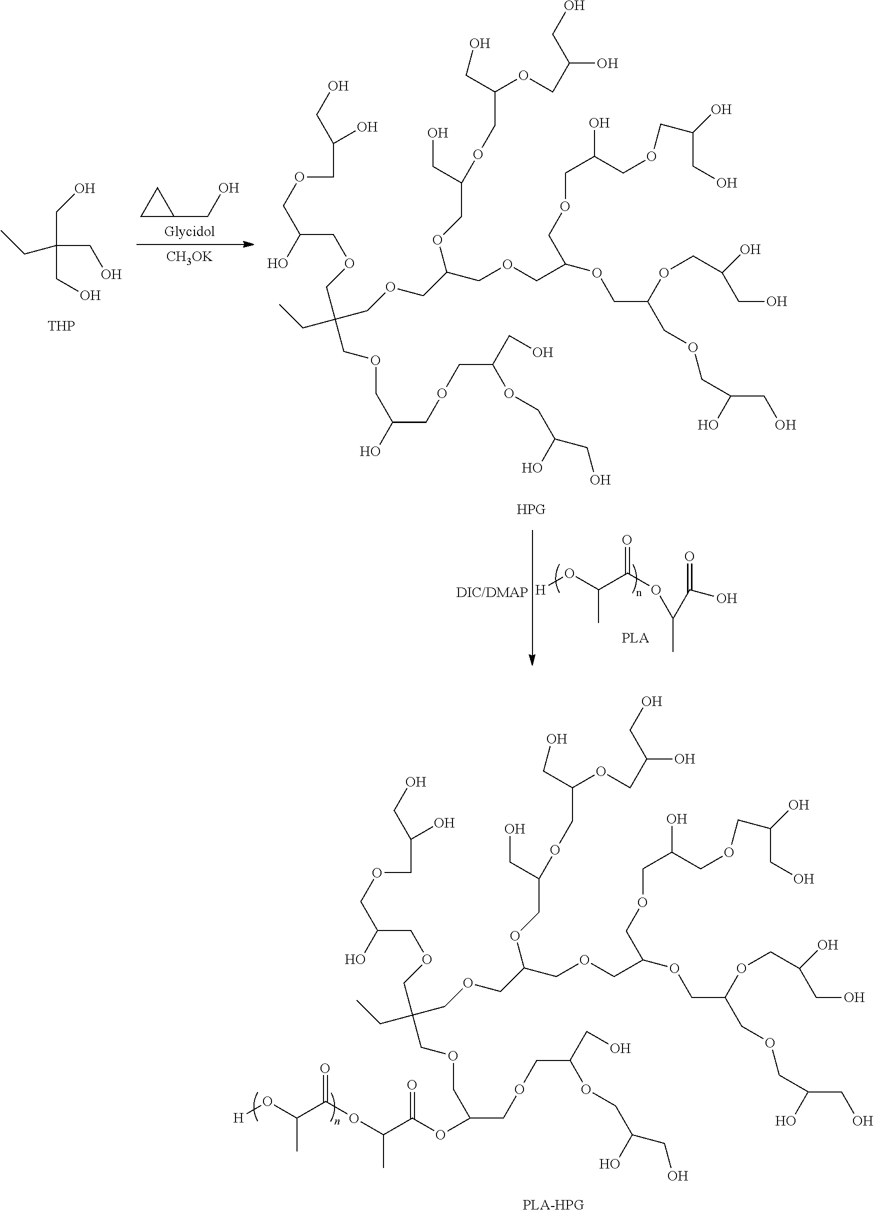

### Modification of Surface Functional Groups

Functional groups on the polymer can be capped to alter the properties of the polymer and/or modify (e.g., decrease or increase) the reactivity of the functional group. For example, the carboxyl termini of carboxylic acid containing polymers, such as lactide- and glycolide-containing polymers, may optionally be capped, e.g., by esterification, and the hydroxyl termini may optionally be capped, e.g., by etherification or esterification.

### Sheddable Polymer Surfaces

The polymers are formed into nanoparticles (preferably 50 nm to under 200 nm), most preferably about 100 nm for maximum diffusion through mucus. The rate of mucus penetration is also a function of the size and density of the hydroxyl groups on the NP surface. In general, higher-density hydroxyl group coatings work the best, and lower molecular weight PEG molecules reduce the likelihood of entanglement of the PEG with mucins, which would hinder diffusion.

In a preferred embodiment, hydrophobic polymer such as PLA is covalently bound to HPG, and used to form the NPs.

Sheddable PEG is created by converting the vicinyl diol groups to aldehydes and then reacting the aldehydes with functional groups on the PEO PEG, such as aliphatic amines, aromatic amines, hydrazines and thiols. The linker has end groups such as aliphatic amines, aromatic amines, hydrazines, thiols and O-substituted oxyamines. The bond inserted in the linker can be disulfide, orthoester and peptides sensitive to proteases.

The hydroxyl groups on the PEG or HPG are shed in the presence of low pH (<5.0), in the presence of large concentrations of competing proteins or enzymes. After the PEG coating is shed, a tissue adhesive rich surface, such as that resulting from exposure of aldehydes, is exposed, allowing for interaction and binding of proteins within the vaginal epithelium, after the hydroxyl groups have facilitated passage through the mucosal and tissue surface.

The HPG can be functionalized to introduce one or more reactive functional groups that alter the surface properties of the particles. For example, hydroxyl-HPG-coated particles have enhanced penetration of mucosa and reduced binding of serum proteins. Such particles are referred to as stealth particle. However, the hydroxyl groups on HPG can be chemically modified to cause the particles to adhere to biological material, such as tissues and cells. Representative functional groups include aldehydes, amines, O-substituted oximes, and combinations thereof.

Other polymers could be used to form the outer surface of the NPs. For example, polyalkylene oxide (PEO) polymers (also referred to as polyalkylenes, polyalkylene glycols, or polyalkylene oxides), preferably branched or dendrimeric, can self-assemble to form particles having a high density of hydroxyl groups on the surface with hydrophobic polymer in the core.

Polyethylene glycol (PEG) is also known as polyethylene oxide (PEO) or polyoxyethylene (POE), depending on its molecular weight. The structure of PEG is commonly expressed as H—(O—CH₂—CH₂)n-OH. PEG, PEO, and POE refer to an oligomer or polymer of ethylene oxide. Branched PEGs have three to ten PEG chains emanating from a central core group. Star PEGs have 10 to 100 PEG chains emanating from a central core group. Comb PEGs have multiple PEG chains normally grafted onto a polymer backbone. PEG may also be used to form PEO dendrimers.

PEG-core dendrimers, branched or star polymers feature excellent solubility in water and a high degree of functionality per PEG chain. Additionally, the terminal amine groups can readily be used in EDC or DCC coupling reactions (after Boc deprotection) with carbonyl-containing compounds, to yield highly functionalized materials for a variety of biomedical applications.

### B. Intravaginal Rings (“IVRs”)

### Reservoirs

Referring to FIGS. 2A and 2B, to extend the duration of the release of drug-loaded NPs in the vagina, the NPs are loaded into individual reservoirs 12 within the matrix 14 within the outer ring around the center hole 16, an otherwise solid IVR 10, preferably made of a non-degradable polymer such as poly(ethylene-co-vinyl acetate) (EVAc). Degradable polymeric “caps” 18 are used to seal each reservoir 10. Caps are formed of polymers that degrade over a period of time so that release occurs over a desired time frame due to the selection of the chemical composition and optionally the diameter and thickness of the caps. In a preferred embodiment, the caps are formed of different molecular weight biodegradable polymers such as PLGA, to provide further control of when NPs are released and at what rate. In some embodiments, the nanoparticles can be formulated in a gel or suppository, which can then be loaded into individual reservoirs of an IVR for placement in the vagina.

### IVRs

IVRs are typically manufactured using a simple injection molding process. Partial or full-length cores or compartments can be made using a temperature-controlled injection molding machine fitted with a custom mold assembly. A preferred formulation, DDU-4320 addition-cured silicone elastomer kit, includes a basic silicone formulation, primarily vinyl-functionalized and hydroxy-terminated poly(dimethyl siloxane)s, is used to make the IVRs containing reservoirs for the NPs.

Since the vaginal ring includes a continuous matrix phase, reservoirs, and caps, the features of each, including the size and number of reservoirs, as well as the release properties of the caps, can be used to control the release of NPs and hence the resulting drug profile. For example, lipid soluble agents (such as estrogens or progestins) can be loaded into the hydrophobic continuous matrix phase. The rate of agent release will depend on the concentration of agent in the matrix, and the polymer used to form the matrix (such as silicone or poly(ethylene-co-vinyl acetate) as well as the molecular weight and composition of the polymer. The rate of nanoparticle release from the reservoirs can be controlled by changing the concentration (or number) of nanoparticles within each reservoir and the degradation rate (hence lifetime) of the degradable polymer cap. The lifetime, and hence release rate, of the composition and molecular wright of the degradable polymer cap can be controlled by selection of the degradable polymer used in the cap, as well as the thickness and crystallinity of the cap material.

In alternative embodiments, the nanoparticles can be formulated in a gel or suppository that can be directly placed in the vagina to release hormone over an extended period of time.

### C. Therapeutic, Prophylactic and Diagnostic Agents

The percent drug loading is typically from about 1% to about 80%, from about 1% to about 50%, from about 1% to about 40% by weight, from about 1% to about 20% by weight, or from about 1% to about 10% by weight. In some embodiments, the percent drug loading is between about 5% and about 50%, or about 10% and about 40%, or about 15% and about 30%. In specific embodiments, drug loading is about 20, 21, 23, 24, 25, 26, 27, 28, 20, or 30%.

Active agents include synthetic and natural proteins (including enzymes, peptide-hormones, receptors, growth factors, antibodies, signaling molecules), and synthetic and natural nucleic acids (including RNA, DNA, anti-sense RNA, triplex DNA, inhibitory RNA (RNAi), and oligonucleotides), sugars and polysaccharides, small molecules (typically under 1000 Daltons), lipids and lipoproteins, and biologically active portions thereof. Suitable active agents have a size greater than about 1,000 Da for small peptides and polypeptides, more typically at least about 5,000 Da and often 10,000 Da or more for proteins. Nucleic acids are more typically listed in terms of base pairs or bases (collectively “bp”).

Preferred compounds include antivirals for treatment or prevention of viral infections such as human papilloma virus (HPV), human immunodeficiency virus (HIV), as well as antibiotics for treatment of sexually transmitted bacterial diseases such as syphilis and gonorrhea, for example, penicillin or azithromycin, which may also be treated with either oral gemifloxacin or injectable gentamicin. Three antiviral medications have been shown to provide clinical benefit in the treatment of genital herpes: acyclovir, valacyclovir and famciclovir.

Hydroxychloroquine (HCQ), a lysosomotropic amine and a hydroxyl derivative of chloroquine, is used for the treatment of acute malaria and autoimmune diseases such as lupus and rheumatoid arthritis. HCQ has been shown to exhibit antibacterial activity both in vitro and in vivo and has demonstrated direct anti-HIV activity by increasing endosomal pH, alteration of enzymes required for gp120 production, and impairment of gp120, integrase, and Tat production. Studies have demonstrated that HCQ can significantly reduce viral loads in HIV-1-infected patients with a CD4 count between 200 and 500 cells/mm³. Furthermore, HCQ has been reported to demonstrate anti-inflammatory and immune modulatory effects.

The following are standard of care therapeutics for various sexually transmitted viral or bacterial diseases.
Nucleoside Reverse Transcriptase Inhibitors: abacavir, didanosine (ddl), lamivudine (3TC), stavudine (d4T), zalcitabine (ddC), zidovudine (ZDV)
Protease Inhibitors: indinavir, nelfinavir, ritonavir, saquinavir, lopinavir plus ritonavir
Nonnucleoside Reverse Transcriptase Inhibitors: delavirdine, efavirenz, nevirapine
Chlamydia: Antibiotics azithromycin, erythromycin, doxycycline
Gonorrhea: Antibiotics: ceftriaxone, cefixime, ciprofloxacin, ofloxacin
Gonorrhea and chlamydia can occur in tandem, if so, it is commonly treated with ceftriaxone plus doxycycline or azithromycin.
Pelvic Inflammatory Disease (PID): Antibiotics: cefotetan or cefoxitin plus doxycycline, clindamycin plus gentamicin, ofloxacin plus metronidazole Typically, two antibiotics are prescribed.
Human Papillomavirus (HPV): imiquimod, podophyllin, podofilox, fluorouracil (5-FU), trichloroacetic acid (TCA), interferon
Genital Herpes: Antivirals: acyclovir, famciclovir, valacyclovir
Syphilis: Antibiotics: penicillin or doxycycline or tetracycline only if allergic to penicillin.

In one preferred embodiment, the agent is of a prophylactic nature, preferably a contraceptive agent. The contraceptive agent can be for female contraception or male contraception. In some embodiments, two or more contraceptive agent are used. The contraceptive agent can be steroidal or non-steroidal.

In some embodiments, the contraceptive agent includes a estrogen formulation, a progestin formulation, or combined estrogen and progestin formulations. Contraceptive agent(s) may be progestogen agents or from progesterone receptor modulators. Progestogen agents, also designated progestins, may be any progestationally active compound. The progestogen agents may be progesterone and its derivatives such as, but not limited to, 17-hydroxy progesterone esters, 19-nor-17-hydroxy progesterone esters, 17-alpha-ethinyltestosterone and derivatives thereof, 17-alpha-ethinyl-19-nor-testosterone and derivatives thereof, norethindrone, norethindrone acetate, ethynodiol diacetate, dydrogesterone, medroxy-progesterone acetate, norethynodrel, allylestrenol, lynoestrenol, fuingestanol acetate, medrogestone, norgestrienone, dimethiderome, ethisterone, cyproterone acetate, levonorgesterol, DL-norgestrel, D-17-alpha-acetoxy-13-beta-ethyl-17-alpha-ethinyl-gon-4-en-3-one oxime, gestodene, desogestrel, DMPA-depo medroxyprogesterone acetate, norgestimate, nestorone and drospirenone. Progesterone receptor modulators may be ulipristal acetate, mifepristone or CDB-4124 or active metabolites thereof.

Preferably, the contraceptive agent is levonorgestrel (LNG), a synthetic progestin having the following chemical structure:

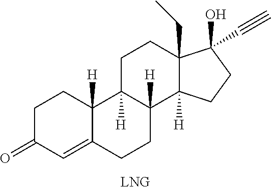

LNG prevents pregnancy by preventing the release of an egg from the ovary or by preventing fertilization of the egg by sperm. LNG may also function by changing the lining of the uterus, thus preventing implantation of a fertilized egg.

Representative anti-cancer agents include, but are not limited to, alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), topoisomerase inhibitors (including camptothecins such as camptothecin, irinotecan, and topotecan as well as derivatives of epipodophyllotoxins such as amsacrine, etoposide, etoposide phosphate, and teniposide), danazole, and combinations thereof. Other suitable anti-cancer agents include angiogenesis inhibitors; receptor tyrosine kinase (RTK) inhibitors such as sunitinib (SUTENT®); tyrosine kinase inhibitors such as sorafenib (NEXAVAR®), erlotinib (TARCEVA®), pazopanib, axitinib, and lapatinib; and transforming growth factor-α or transforming growth factor-O inhibitors.

Other therapeutics that may be delivered with this technology include antibiotics (antibacterial, antiviral or antifungal), and antiinflammatory (such as steroids like cortisone and prednisone and non-steroidal antiinflammatories such as naproxan).

Imaging agents such as radioopaque compounds may also be incorporated to facilitate localization at the time of placement or removal.

### III. Methods of Making Compositions

### A. Method of Making NPs with Sheddable PEG Coating

The preferred method demonstrated in the examples is a type of self-assembly of polymers to form nanoparticles referred to as direct dissolution followed by ultrasonication. However, solvent evaporation/nanoprecipitation followed by ultrasonication can also be used and may be preferred for the encapsulation of drugs. Ultrasonication is very important to obtaining monodisperse nanoparticles.

Methods for making nanoparticles are known. See, for example, Teo B. M. (2016) Ultrasonic Synthesis of Polymer Nanoparticles. In: Handbook of Ultrasonics and Sonochemistry. Springer, Singapore. https://doi.org/10.1007/978-981-287-278-4_14. Nanoprecipitation is a simple method used for encapsulation of both hydrophilic and hydrophobic drugs in nanoparticles. The method results in instantaneous formation of nanoparticles, is an easy to perform technique, can be easily scaled up and is a one-step procedure. The method requires addition of two solvents that are miscible with each other and results in spontaneous formation of nanoparticles on phase separation. The first solvent is the one in which the polymer and the drug dissolves but not in the second non-solvent. A modified nanoprecipitation method utilizes a co-solvent to either increase the entrapment efficiency of the drug in nanoparticles or to reduce the mean particle size of the nanoparticles.

See also Zielińska, et al., Molecules 2020, 25, 3731, reviewing methods for production of polymeric nanoparticles. In general, two main strategies are employed, namely, the dispersion of preformed polymers or the polymerization of monomers. In order to load compounds in polymeric NPs, techniques based on the polymerization of monomers allow insertion with greater efficiency and in a single reaction step. Regardless of the method of preparation employed, the products are usually obtained as aqueous colloidal suspensions.

Solvent evaporation was the first method developed to prepare polymeric NPs from a preformed polymer. In this method, the preparation of an oil-in-water (o/w) emulsion is initially required, leading to nanosphere production. First an organic phase is prepared, consisting of a polar organic solvent in which the polymer is dissolved, and the active ingredient (e.g., drug) is included by dissolution or dispersion. Then an aqueous phase which contains a surfactant such as polyvinyl acetate, PVA, is prepared. The organic solution is emulsified in the aqueous phase with a surfactant, and then it is typically processed by using high-speed homogenization or ultrasonication, yielding a dispersion of nanodroplets. A suspension of NPs is formed by evaporation of the polymer solvent, which is allowed to diffuse through the continuous phase of the emulsion. The solvent is evaporated either by continuous magnetic stirring at room temperature (in case of more polar solvents) or in a slow process of reduced pressure (as happens when using dichloromethane and chloroform). After the solvent has evaporated, the solidified nanoparticles can be washed and collected by centrifugation, followed by freeze-drying for long-term storage. This method allows the creation of nanospheres.

Emulsification/Solvent Diffusion consists of the formation of an o/w emulsion between a partially water-miscible solvent containing polymer and drug, and an aqueous solution with a surfactant. The internal phase of this emulsion consists of a partially hydro-miscible organic solvent, such as benzyl alcohol or ethyl acetate, which is previously saturated with water in order to ensure an initial thermodynamic balance of both phases at room temperature. The subsequent dilution with a large amount of water induces solvent diffusion from the dispersed droplets into the external phase, resulting in the formation of colloidal particles. Generally, this is the method used to produce nanospheres, but nanocapsules can also be obtained if a small amount of oil (such as triglycerides: C6, C8, C10, C12) is added to the organic phase. Depending on the boiling point of the organic solvent, this latter stage can be eliminated by evaporation or by filtration. This method can yield NPs with dimensions ranging from 80 to 900 nm.

Emulsification/Reverse Salting-Out is a modification of the emulsification/reverse salting-out method. The salting-out method is based on the separation of a hydro-miscible solvent from an aqueous solution, through a salting-out effect that may result in the formations of nanospheres. The main difference is the composition of the o/w emulsion, which is formulated from a water-miscible polymer solvent, such as acetone or ethanol, and the aqueous phase contains a gel, the salting-out agent and a colloidal stabilizer. Examples of suitable salting-out agents include electrolytes, such as magnesium chloride (MgCl₂), calcium chloride (CaCl₂) or magnesium acetate [Mg(CH₃COO)₂], as well as non-electrolytes e.g., sucrose. The miscibility of acetone and water is reduced by saturating the aqueous phase, which allows the formation of an o/w emulsion from the other miscible phases. The o/w emulsion is prepared, under intense stirring, at room temperature. Then the emulsion is diluted using an appropriate volume of deionized water or of an aqueous solution in order to allow the diffusion of the organic solvent to the external phase, the precipitation of the polymer, and consequently, the formation of nanospheres. The remaining solvent and salting-out agent are eliminated by cross-flow filtration. The dimensions of the nanospheres obtained by this method vary between 170 and 900 nm. The average size can be adjusted to values between 200 and 500 nm, by varying polymer concentration of the internal phase/volume of the external phase. As the solvent diffuses out from the nanodroplets, the polymer precipitates in the form of nanocapsules or nanospheres.

Nanoprecipitation is a method frequently used for the preparation of polymeric NPs with around 170 nm dimensions, but it also allows the acquisition of nanospheres or nanocapsules. Nanospheres are obtained when the active principle is dissolved or dispersed in the polymeric solution. Nanocapsules are obtained when the drug is previously dissolved in an oil, which is then emulsified in the organic polymeric solution before the internal phase is dispersed in the external phase of the emulsion.

In solvent evaporation, the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid nanoparticles. The resulting nanoparticles are washed with water and dried overnight in a lyophilizer. Nanoparticles with different sizes (0.5-1000 nms) and morphologies can be obtained by this method. This method is useful for relatively stable polymers like polyesters and polystyrene.

The technique to be used can depend on a variety of factors including the polymer used to form the particles, the desired size range of the resulting particles, and suitability for the therapeutic, diagnostic, and/or prophylactic agent to be incorporated.

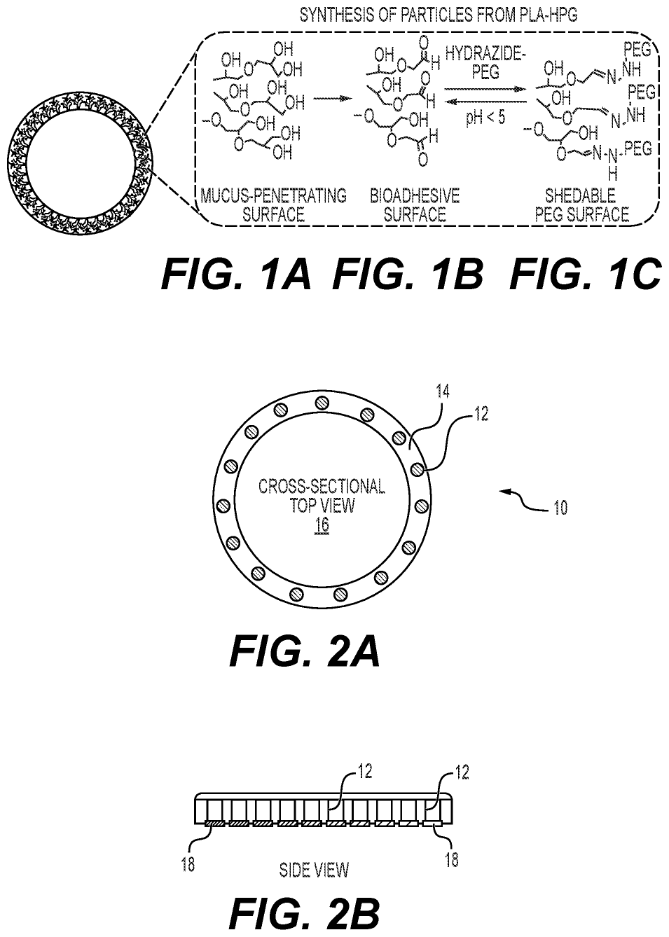

The HPG-coated NPs, formed using standard techniques such as emulsion or phase separation, are modified by forming the NPs using hydrophobic polymer such as polylactide-co-glycolide covalently attached to a PEO such as PEG or HPG, which orients to the surface during NP formation under appropriate conditions. (FIG. 1A) Sheddable PEG is created by converting the vicinyl diol groups to aldehydes and then reacting the aldehydes with functional groups on the PEO PEG, such as aliphatic amines, aromatic amines, hydrazines and thiols. HPG surface functional groups are oxidized to tissue adhesive groups such as aldehyde terminated PLA-HPG_{ALD} NPs and conjugated to hydrazide terminated PEG (NH₂NH-PEG) by a reversible Schiff base bond. (FIG. 1B) The linker has end groups such as aliphatic amines, aromatic amines, hydrazines, thiols and O-substituted oxyamines. The bond inserted in the linker can be disulfide, orthoester and peptides sensitive to proteases.

PEG with a functional group or a linker can form a bond with aldehyde on PLA-HPG_{ALD} and reverse the bioadhesive (sticky) state of PLA-HPG_{ALD} to stealth state. This bond or the linker is labile to pH change, high concentration of peptides, proteins, and other biomolecules and/or enzymes. After administration systematically or locally, the bond attaching the PEG to PLA-HPG_{ALD} is cleaved to release the PEG. Cleavage occurs in response to environment, which may be water (i.e., hydrolysis), low pH due to tumors or infection, or enzymatic (one of the many esterases and other enzymes found in the tissue. (FIG. 1C) With the PEG removed, adhesive groups such as aldehydes and amines binding to reactive groups on the tissue, to adhere the particles at and/or into the tissue or extracellular materials such as proteins.

The percent drug loading is typically from about 1% to about 80%, from about 1% to about 50%, from about 1% to about 40% by weight, from about 1% to about 20% by weight, or from about 1% to about 10% by weight. In some embodiments, the percent drug loading is between about 5% and about 50%, or about 10% and about 40%, or about 15% and about 30%. In specific embodiments, drug loading is about 20, 21, 23, 24, 25, 26, 27, 28, 20, or 30%.

### B. Method of Making IVR Containing NPS

A number of methods are known for making intravaginal rings (IVR). See, for example, US2020-0054553-A1. These generally fall into two categories: molding optionally utilizing extrusion, heating of a film, and/or solvent casting. Cores or compartments can be formed in the molded rings. For the more complex structures described herein, three dimensional (3D) printing, lithography or other 3D additive process, either to make the device per se or to make a mold which includes the complex structures are used. For example, molds can be 3D printed using a commercially available printer. Representative dimensions of the mold cavities are 4.2 mm diameter×1.4 mm depth. The final product may be further modified using a process such as film or spray coating.

IVRs have been made with a range of dimensions (diameter of the ring ‘loop,’ and the smaller diameter of the ring material cross-section). Data are published for both the dimensions and the vaginal position of these devices, the latter based on MRI images. For example, the NUVARING® is a flexible, transparent, colorless, contraceptive ring, made of ethylene vinyl acetate copolymers and magnesium stearate, measuring 54 mm in diameter with a 4 mm cross-section. Sagittal MRI of the NUVARING® in vivo show that the most superior portion of the ring rests within the fornix behind the cervical os. Axial MRI images reveal that the vaginal tissues compress the NUVARING® into a 57 mm×50 mm oval. These images shows that the entire ring is in contact with vaginal tissues, and therefore ring diameter can be used to approximate the width of the vaginal canal at about 50 mm.

### C. Method of Making IVR with Reservoirs

IVRs with reservoirs can be produced by a number of methods. For example, IVRs from EVAc polymer resins can be made using injection molding. Briefly, melted EVAc polymer resin is injected into a mold using established techniques. Metal molds are fabricated for devices of the desired size: each mold must also include cylindrical protrusions to produce the empty reservoirs in the IVR ready for NP loading: 30 reservoirs are appropriate for human devices, although other reservoir numbers may be useful. IVR dimensions are well known in the field. Human IVRs are approximately 56 mm (outer diameter), by 40 mm (inner diameter), and 6-8 mm (cross-sectional diameter). Reservoirs are equally spaced, with an inner diameter of 2-3 mm.

### D. Methods of Loading and Sealing Reservoirs in IVR

The reservoirs in the IVR can be filled with NPs using a variety of techniques, with varying degrees of automation. A simple method is to employ nanoparticles at high concentration in an aqueous phase, which is quickly dispensed into each of the reservoirs, and then the whole device is flash-frozen and the solvent removed by lyophilization.

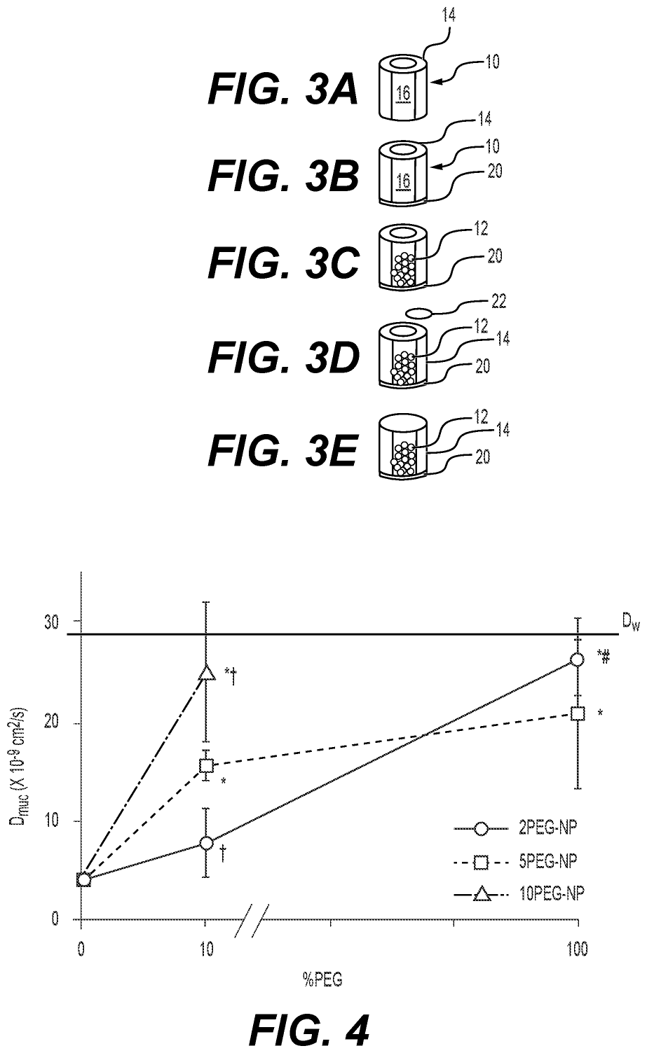

FIGS. 3A-3E are diagrams of the assembly of the NP filled reservoirs 12. In the first step, reservoirs 12 are created within the matrix 14 of the IVR ring structure 10. The reservoirs 12 include the wall formed by the matrix 14, the center portion of the reservoirs 12 containing the NPs, the degradable bottom cap 20 of the reservoir 12 within the matrix 14, and the non-degradable cap 22. As shown in FIG. 2A, each of the 15 reservoirs 12 is sealed with a degradable polymer capping layer 20 where a different molecular weight of PLGA polymer is used on each of the caps 20, allowing time-dependent activation.

FIGS. 3A-3E show schematically the step-by-step fabrication of NP-loaded reservoirs in IVR. FIG. 3A, an empty reservoir 12 is (FIG. 3B) sealed on the bottom with a degradable polymer cap 20; (FIG. 3C), the reservoir 12 is filled with NPs and then (FIG. 3D) sealed on the top by a non-degradable polymer layer cap 22, resulting in the final filled reservoir 12 in the IVR 10 (FIG. 3E).

### E. Coating of IVR

The IVR can include a coating of a degradable polymer like poly(PDL-co-DO) or a polyester such as PLA or PLGA, or a film. The IVR can include a core, or a combination thereof. The coating and the core can be drug-containing or drug-free. For example, IVR can contain a drug-free (also referred to herein as “pure polymer”) core to shorten the final drug release tail. Additionally, or alternatively, the IVR can include a drug-free (i.e., pure polymer) coating to reduce initial burst release.

In an exemplary method for pure polymer core fabrication, polymer is loaded inside a baking mold (e.g., (d=1 mm)) and baked to create a pure polymer core.

Coated implants can be fabricated by preparing a polymer sheet and coating the polymer sheet on to the IVR. In an exemplary method for polymer sheet fabrication, a polymer sheet is formed by dissolving PDL-co-DO in chloroform, pouring the solution into a glass dish, evaporating the chloroform (e.g., over night at room temperature), and harvesting the PDL-co-DO sheet.

In an exemplary method for coating polymer sheets onto IVR, an IVR is sandwiched between two polymer sheets and placed inside a baking mold and allowed to bake (e.g., for 10 min at 70-80° C. in atmosphere pressure with argon protection).

In preferred embodiments, the coating is drug-free and is effective to reduce any initial burst effect of drug released from the implant relative to an uncoated IVR.

After coating, excess polymer can be removed from the IVR.

### IV. Methods of Use

The IVR devices described herein are placed in, and removed from, a woman the same as other intravaginal devices. The period of time prior to removal depends on the drugs which are delivered, and whether the use is therapeutic or prophylactic.

The IVR can be used for the delivery of therapeutic and/or prophylactic agents that include, but are not limited to, anti-inflammatory drugs, anti-proliferatives such as anti-cancer agents, anti-infectious agents, such as antibacterial, antiviral, and anti-yeast agents, and contraceptives or fertility enhancing agents. These can be used to prevent pregnancy, enhance fertility, treat disorders such as endometriosis, fibroids, and tumors, as well as to prevent and/or treat infection, especially with retrovirus and DNA virus, or help restore normal uterine physiology, for example, disrupted by overgrowth of yeast or non-lactobacillus species of bacteria. These can also be used to prevent and/or treat bacterial infections caused by Chlamydia thrachomatis, Neisseria gonorrhoeae and Treponema pallidum. Additionally, the particles can be used for the controlled release of bioactive agents for providing post-menopausal hormone replacement therapy (HRT). The dosage is determined using standard techniques based on the drug to be delivered and the method and form of administration.

A prophylactic use is typically when the IVR releases drug in one or more time period over a prolonged period of time, for example, to prevent infection with a virus such as human papilloma virus (HPV) or human immunodeficiency virus (HIV) or other infectious agent, including species of bacteria which are not present in significant amounts in the healthy vagina, or contraceptive to prevent pregnancy or endometrial overgrowth (endometriosis). Therapeutic indications can include delivery of chemotherapeutic agents such as those used to decrease or destroy fibroid tissues or various types of tumors.

The timing of release and amount released would be that amount which is prophylactically or therapeutically effective, and may range from 2-3 weeks to a year or more. Release may occur from one or more reservoirs, as needed, and at such intervals as required to produce the effective local or systemic dosage. In a preferred embodiment, the dosage is that which is effective locally (or regionally, that being the pelvic region).

The present invention will be further understood by reference to the following non-limiting examples.

### Example 1: Manufacture of IVR with Complex Geometrics

Successful development of IVRs for the delivery of drugs requires the selection of an appropriate polymer. In the case of hydrophilic drugs, the ideal polymer for IVR fabrication should satisfy several conditions. First, it should provide high drug solubilization capacity so that a sufficient quantity of the drug can be incorporated into the IVR formulation. Second, the polymer should be able to control the drug release based on properties such as water swellability and drug diffusion within the polymeric matrix. Last, its mechanical and chemical properties should allow the IVR to remain within the vaginal lumen without eliciting tissue damage, and the selected polymer should be stable in the acidic vaginal environment (pH, 3.5-4.2), due to the lactic acid present in the vagina.

### Example 2: Diffusion of PEG-PLA Mucus-Penetrating NPs in Human Cervical Mucus

### Materials and Methods

### Synthesis of NPs

PLA-PEG NPs were made by single emulsion. 50 mg PLA-PEG copolymer dissolved in 1.5×10³ ml solvent mixture (Ethyl acetate:DMSO, 4:1) was added into 4 ml DI water with 2.5% PVA under vortexing and then subjected to probe sonication for 3 cycles of 10 s each. The resulting emulsion was diluted in 20 ml DI water with 0.1% Tween 80 under stirring. The emulsion was stirred for at least 5 h or hooked up to a rotavapor to evaporate the ethyl acetate and then the solution was applied to an AMICO™ ultra centrifuge filtration unit (100 k cut-off). The NPs were washed by filtration for two times then suspended in a 10% sucrose solution.

Particles in 1×PBS at 0.5 mg/mL were added to capillary borosilicate glass tubing (VITROCOM®) filled with fresh human cervical mucus and sealed with CRITOSEAL® (Krackeler Scientific). The fluorescence profile of the mucus/solution interface was immediately taken with a Zeiss microscope under green fluorescent filter using a 20° ø objective and recorded as time 0. The entire tube was kept stationary and in the dark until the next measurement. Fluorescence intensity profile from interface was taken using ImageJ software with the length scale calibrated to a known distance. Background fluorescence was subtracted and the entire profile normalized to maximum intensity. Three sample curves were taken for each tube at given time point, two tubes (duplicates) were used for each particle sample (total n) 6).

### Calculation of Diffusion Coefficients

Particle diffusion coefficients in water (Dw) were calculated using the Stokes-Einstein equation for particle population. The fluorescent profile of particles from the mucus-solution interface into the gel was recorded and fitted to a solution to diffusion model based on Fickian mass transport in a semi-infinite medium using numerical integration by finite difference method. The setup and mathematical methods to solve for sample diffusion coefficients are described in detail by Radomsky et al., Biomaterials 11:619-624 (1990), where diffusion of various fluorescently labeled probes, antibodies and proteins was similarly observed in human cervical mucus. The measured diffusion profile was fitted to a governing equation, commonly known as Fick's second law: where the spread of particle concentration over time (∂C/∂t) depends on its effective diffusion coefficient (D, in this case representing Deff or Dmuc) and the second derivative of the concentration (∂2C/∂x2).

### Results and Discussion

Adding stealthy PEG molecules to PLGA nanoparticles facilitated the production of nanoparticles that readily penetrated the mucus. This mucus-penetrating ability depended on controlling the size and density of PEG on the nanoparticle surface. FIG. 4 is a graph showing diffusion of the mucus-penetrating nanoparticles in the human cervical mucus. NPs that contained high densities of low molecular weight PEG units (density increases are recorded on the x-axis) diffused as fast in mucus as in water. MW of PEG (2K—circles, 5K—squares, and 10K—triangles) were tested for their affect on the rate of diffusion.

Fully PEGylated particles (100% PEG) yielded the highest diffusion coefficients while partially PEGylated particles (10% PEG) exhibited an intermediate range of diffusion. The molecular weights of the PEG particles also affected the diffusion rate of the PEG particles through the mucus. For example, the partially PEGylated particles (10% PEG) diffused significantly faster for formulations containing 10 and 5 kDa PEG but not for those partially coated by 2 kDa PEG. However, fully coated (100%) nanoparticle formulations with 2 kDa PEG diffuse significantly faster than formulations partially coated (10%) with 2 kDa PEG. These results show that higher-density PEG coatings and lower molecular weight PEG molecules reduced the likelihood of entanglement of the PEG with mucins, which would hinder diffusion.

### Example 3: Synthesis and Characterization of the Stealth, Aggregation-Resisting, Mucus-Penetrating, and Local Retention Properties of PLA-HPG Nanoparticles

### Methods and Materials

PLA-HPG NPs were made as described by Deng, et al. Biomaterials 35(24):6595-602 (2014). Epub 2014 May 9. doi: 10.1016/j.biomaterials.2014.04.038.

Fifty mg of PLA-HPG copolymer dissolved in 1.5×10³ ml solvent mixture (Ethyl acetate:DMSO 4:1) was added into 4 ml deionized (“DI”) water under vortexing and then subjected to probe sonication for 3 cycles at 10 s each. The resulting emulsion was diluted in 20 ml DI water under stirring. It was stirred for at least 5 h or hooked up to a rotavapor to evaporate the ethyl acetate and then applied to an AMICO™ ultra centrifuge filtration unit (100 k cut-off). The NPs were washed by filtration 2 times then suspended in a 10% sucrose solution. The NPs were kept frozen at 20° C.

The NPs were examined by Transmission electron microscopy.

NPs were also prepared containing drug and release measured in vitro over a period of a week, and correlated with size of the NPs.

Release was compared for the PLA-PEG NPs of Example 1 with the PLA-HPG Nps.

### Results and Discussion

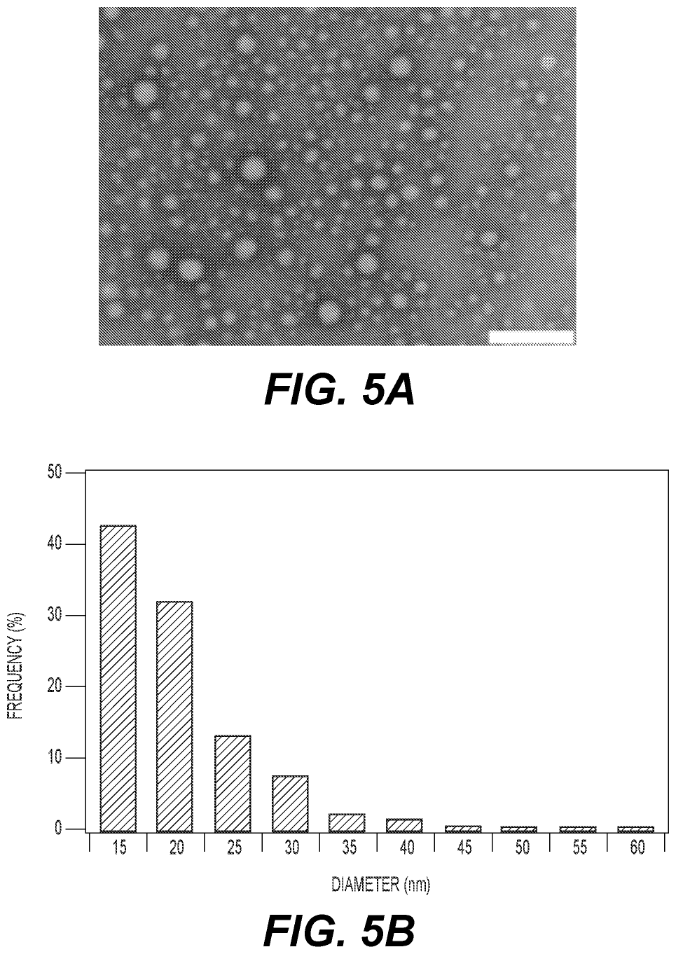

A method for improving the aggregation-resisting, mucus-penetrating and local retention properties of nanoparticles was created. Poly(lactic acid) (PLA) was conjugated to hyperbranched polyglycerols (HPG) to produce PLA-HPG nanoparticles. TEM confirmed the spherical shape of the PLA-HPG nanoparticles. See FIG. 5A. Most of the PLA-HPG nanoparticles were 15-20 nm in diameter, with ˜40% of the nanoparticles being 15 nm and ˜30% of the nanoparticles being 20 nm. See FIG. 5B

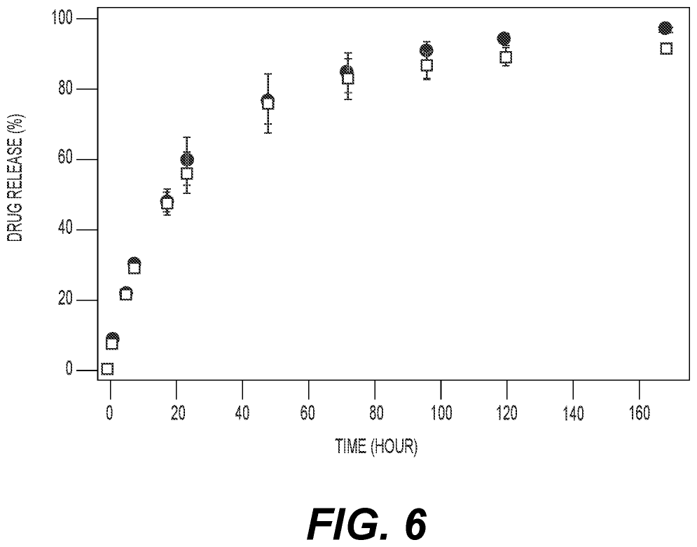

No differences were observed in the patterns of drug release. FIG. 6 compares the rate of drug release from PLA-HPG and PLA-PEG nanoparticles. More than half of the drug (˜59% and ˜56%) was released from the PLA-HPG and PLA-PEG nanoparticles, respectively, following 24 hours of incubation. The drug was released over a period of one week from both the PLA-HPG and PLA-PEG formulations.

The nanoparticles resist aggregation and are readily resuspended to free particles from the solid state. The appearance of emulsions of PLG-HPG, PLA-HPG/drug and PLA-PEG/drug nanoparticles were compared after 30 min, 1 day and 10 days. Once suspended in fluids, the PLA-HPG nanoparticles remain singly suspended in solution longer than PLA-PEG/drug nanoparticles. These results show that PLA-HPG/drug nanoparticles have greater stability in suspension than PLA-PEG/drug nanoparticles.

The PLA-HPG nanoparticles demonstrate better stealth than PLA-PEG nanoparticles, the current gold standard for stealth coating. The HPG coating substantially enhanced the stealthiness of the nanoparticles by extending the elimination half-life for PLA-HPG to 10 hours compared to 6 hours for PLA-PEG.

The multiple hydroxyl groups on HPG enable the attachment of multiple ligands onto the HPG corona. Importantly, the majority of the hydroxyl groups on HPG are vicinal diols. These vicinal diols on PLA-HPG nanoparticles can be easily oxidized to aldehyde groups that allow attachment of many kinds of ligands under mild conditions.

### Example 4: Creation of Nanoparticles with a Sheddable PEG Coating

### Materials and Methods

To create nanoparticles with a sheddable PEG coating, the PLA-HPG nanoparticles of Example 2 were oxidized to aldehyde terminated PLA-HPG_{ALD} nanoparticles and conjugated to hydrazide terminated PEG (NH₂NH-PEG) by a reversible Schiff base bond. Although this Schiff base bond is more stable than the Schiff base between an amine (such as N-terminal or lysine side chain of proteins) and an aldehyde, it is labile at pH<5.0 and in the presence of large concentrations of competing proteins.

NPs were administered intravaginally to mice and retention assessed over time.

### Results and Discussion

Data from in vivo experiments in mice demonstrated that sheddable PEG nanoparticles (SPNPs) have significantly longer retention times following vaginal delivery than either conventional mucus-penetrating nanoparticles (with stable PEG coatings, MPNPs) or bioadhesive nanoparticles (BNPs). The superior performance of the SPNPs is attributed to their ability to penetrate mucus and become converted to a bioadhesive form upon contact with epithelial cells, after which they are immobilized and retained for long periods. In contrast, the conventional mucus-penetrating nanoparticles (MPNPs), which never become adhesive, are cleared with by natural turnover of mucus or lymphatic drainage if they reach the tissue space. Conventional bioadhesive nanoparticles (without PEG surfaces) stuck to mucus instead of penetrating it and were cleared by mucus turnover. For example, one out of two mice had substantial retention of the SPNPs at 48 hours (2 days). This observation was not made with nanoparticles that are PEGylated by conventional methods. These data demonstrate the utility of sheddable PEG nanoparticles for prolonged delivery of agents following intravaginal delivery.

### Example 5: PLA-HPG Nanoparticles can be Loaded into Reservoirs in EVAc Devices and Released

### Materials and Methods

Methods for loading PLA-HPG nanoparticles into millimeter-scale reservoirs within solid Ethylene vinyl acetate copolymer (EVAc) reservoirs were developed and tested. EVAc containing a single reservoir were used to test for feasibility. The nanoparticles were loaded into reservoirs as solids.

For an uncapped reservoir, the device was immersed in water and the nanoparticles were released over a short period of time (˜10 min).

The NPs were loaded into EVAc chambers and submerged in water. TEM images were prepared prior to loading and after ten minutes of incubation in a 37° C. shaker.

Dynamic light scattering (DLS) was used to measure hydrodynamic diameters of the particle population.

The released nanoparticles were stably suspended in the immersion medium for at least 10 hours. Similar experiments were performed with conventional nanoparticles, PLGA-PEG nanoparticles as described in Example 1.

### Results and Discussion

Prior to drying and loading into the reservoirs, the PLA-HPG NPs had a mean diameter of 86 nm. The mean diameter of the particles' following release was approximately 98 nm and therefore, not significantly different from the particle diameter prior to loading into the reservoirs.

In these experiments, very little PLA-PEG NPs are released from the reservoirs and, any nanoparticles that are released from the reservoirs are highly aggregated or destroyed by the fabrication process.

This is in contrast to the ability of the PLA-HPG NPs to be loaded into a device reservoir and released as free nanoparticles, allowing them to be incorporated into intravaginal rings that slowly release nanoparticles for longer duration of action upon drug delivery.

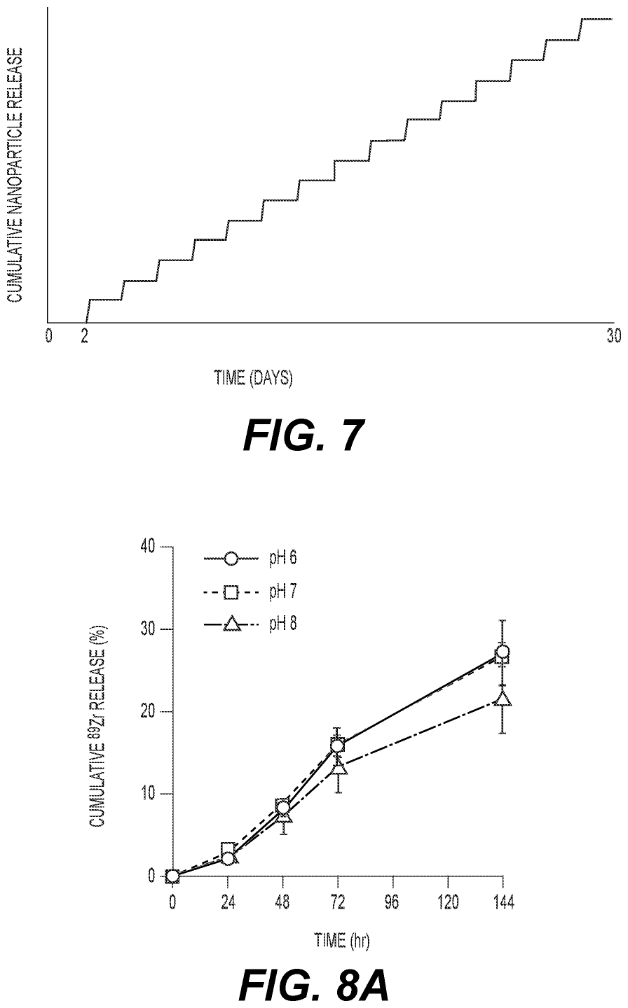

FIG. 7 is a graph of predicted cumulative NP release over time (hours) showing how the reservoirs and NPs can be used to achieve long term delivery.

### Example 6: Kinetics and Safety of Vaginally Administered Bioadhesive Nanoparticles in Cynomolgus Monkeys

Long-lasting vaginal dosage forms could improve the therapeutic efficacy of vaginal microbicides, but achieving long-term delivery to the vaginal canal has been a significant challenge. As described in the examples above, a bioadhesive nanoparticle (BNP) formulation that is retained in the vaginal canal of mice for several days was developed. Further studies were conducted to evaluate the retention and safety of BNPs in non-human primates.

### Materials and Methods

Abbreviations PEG, polyethylene glycol; PLA, polylactic acid; HPG, hyperbranched polyglycerol; BNP, bioadhesive nanoparticle; NHP, non-human primate; PET, positron emission tomography; DFO, deferoxamine; TLC, thin liquid chromatography; ROI, region of interest; TOF, time-of-flight; PSF, point spread function; OSEM, ordered subset expectation maximization; BDL, below detection limit; PVE, partial-volume effect; PBS, phosphate buffered saline.

### Nanoparticle Preparation and BNP Conversion

Non-adhesive PLA-HPG nanoparticles (NNPs) were prepared using a single emulsion, solvent evaporation process. One hundred (100) mg of polymer was dissolved overnight in 2.4 mL of ethyl acetate. The next morning, 0.6 mL of DMSO was added to the polymer solution, and the polymer solution was added dropwise to 4 mL of deionized water while vortexing. The emulsion was placed on ice and sonicated (3×10 s with 10 s on ice in between) using a probe sonicator. The solution was mixed with 10 mL deionized water and transferred to a round-bottom flask, and organic solvent was removed using a rotary evaporator for 30 min. NNPs were then transferred to a centrifugal filter (Amicon Ultra-15, 100 kDa MWCO, Sigma Aldrich) and centrifuged three times at 4000×g to wash away excess polymer and solvent. After the final spin, nanoparticles were resuspended in deionized water and were flash frozen in liquid nitrogen and stored at −20° C. until use. FITC-loaded nanoparticles were prepared by substituting 10 mg of PLA-HPG with FITC-PLA (PolySciTech AV039). To prepare BNPs, NNPs were diluted to a concentration of 25 mg/mL, mixed with equal volumes of 10×PBS and 0.1 M NaIO₄, and incubated on ice. After 20 min, one volume of 0.2 M Na₂SO₃ was added to quench the reaction. BNPs were then transferred to a centrifugal filter and washed three times at 4000×g.

### ⁸⁹Zr-BNP and ⁸⁹Zr-DFO Preparation

BNPs were labeled with ⁸⁹Zr by conjugating a chelator, deferoxamine mesylate (DFO, CAS #138-14-7, Sigma) to the nanoparticle surface. Surface conjugation of DFO was achieved through reductive amination. After BNP conversion, BNPs (25 mg/mL) were incubated with 1 molar equivalent of DFO mesylate for 4 hrs at room temperature. 40 molar equivalents of NaCNBH₃ were added, and BNPs were incubated for an additional 40 hrs. DFO-conjugated BNPs were washed four times to remove excess DFO mesylate and NaCNBH₃ and resuspended at a concentration of 25 mg/mL. DFO-BNPs were then labeled with neutralized ⁸⁹Zr-oxalate in 0.25 M HEPES (pH 7.4) at a molar activity of 727 MBq/mol and were incubated for 30 min at room temperature. Radiochemical yield was determined via radio-thin liquid chromatography (radio-TLC), ⁸⁹Zr-BNPs were washed three times using a centrifugal filter (Amicon Ultra-0.5, 100 kDa MWCO, Sigma Aldrich), and resuspended to a final concentration of approximately 222 MBq/mL (decay-corrected to the time of delivery). To assess uptake without the BNPs, ⁸⁹Zr-DFO was prepared; 0.1 mg/mL DFO mesylate was labeled with neutralized ⁸⁹Zr-oxalate in 0.25 M HEPES (pH 7.4) at a molar activity of 2070 MBq/mol and was incubated for 30 min at room temperature. Radiochemical yield was determined via radio-TLC. ⁸⁹Zr-DFO was then loaded into an activated SEP-PAK PLUS C18 cartridge (Waters Corp), washed twice with deionized water, and eluted with 95% ethanol. Excess ethanol was evaporated at 90° C. for 1 hr, and ⁸⁹Zr-DFO was resuspended to a final concentration of approximately 222 MBq/mL, decay-corrected to the time of delivery.

### In Vitro Release

⁸⁹Zr release from BNP conjugates was assessed in vitro. After conjugation, ⁸⁹Zr-BNPs were diluted into simulated vaginal fluid at a pH of 5, 6, or 7 in order to cover the range of vaginal pH levels seen in humans and cynomolgus monkeys, and they were then incubated at 37° C. and 500 rpm. At pre-determined time points (1, 2, 3, 6 days), ⁸⁹Zr released from BNPs with a centrifugal filter (Amicon Ultra-0.5, 100 kDa MWCO, Sigma Aldrich). The filtrate activity was measured using a Hidex AMG automatic gamma counter (Hidex, Turku, Finland). Nanoparticles were resuspended in the corresponding simulated vaginal fluid and placed back on the incubator for the subsequent measurement.

### NHP Study Plan and Population

All NHP experiments were conducted under a protocol approved by the Yale University Institutional Animal Care and Use Committee. Two female cynomolgus monkeys (age, 11.2±1.1 y; weight, 6.1±1.5 kg) were used in the PET imaging study and the toxicity study. For the toxicity study, both animals were treated with nanoparticles, and untreated tissue was acquired for histological comparison from the Wisconsin National Primate Research Center (WNPRC) Nonhuman Primate Biological Materials Distribution (NHPBMD) core. All animals were injected with 30 mg of depo-provera (medroxyprogesterone acetate) 25-30 days prior to the start of every study. Animals were given at least 4 weeks to recover between experiments.

### PET Imaging and Quantification of Radioactivity in the Blood

⁸⁹Zr-BNPs (108 MBq) or ⁸⁹Zr-DFO (100.6 MBq) were administered intravaginally on Day 0 to two monkeys using a 1 mL syringe prior to scanning on a Siemens Biograph mCT scanner. List-mode PET data were acquired firstly in a stationary bed position over the animal's pelvis for 30 min, followed by 90 min of continuous-bed-motion whole-body imaging, for a total of 120 min. PET scans were repeated on subsequent days using the same scanning paradigm. For the ⁸⁹Zr-BNP study, PET scans were taken on days 0, 1, and 5. For the ⁸⁹Zr-DFO study, PET images were acquired for 120 min after administration. PET images were reconstructed using time-of-flight (TOF)+point spread function (PSF) modeling with an ordered subset expectation maximization (OSEM) algorithm with 2 iterations of 21 subsets. CT images were acquired in addition to PET scanning for attenuation and scatter correction, and for anatomical delineation. To improve CT delineation, 3 mg/mL of contrast (Omnipaque 300, GE Healthcare) was injected at a rate of 40 mL/min, and the image was acquired 1 min after the start of contrast injection. Blood samples were collected in heparinized tubes before and after each scan for activity measurements using a Hidex AMG automatic gamma counter (Hidex, Turku, Finland). Regions of interest (ROIs) were manually defined on the CT image for the following regions: vagina, uterus, bladder, kidneys, spleen, stomach, liver and heart. Radioactivity concentration within the region of interest was averaged throughout the PET scan and is reported in units of Bq/mL.

### Vaginal Safety

To assess the safety of BNPs for vaginal application, 0.5 mL of FITC-BNPs (100 mg/mL in PBS) were applied topically to the vagina using a 1 mL syringe four times over the course of one week (days 0, 2, 4, and 7). For cytokine analysis, vaginal fluid and blood samples were collected 5 days before the start of the first delivery, and then immediately prior to nanoparticle application on each delivery day. To collect vaginal fluid, pre-weighed Weck-Cel spears (Beaver Vistec International) were inserted partway into the vaginal canal and left in place for 5 min. The spears were then reweighed to calculate absorbed vaginal fluid and stored at −80° C. until use. To prepare vaginal fluid samples for cytokine analysis, the swab tip was cut from the handle and placed in the top of a Spin-X centrifugal filter (0.22 μm, Corning). 300 μL of elution buffer (PBS; 0.25% BSA; 1:100 dilution of protease inhibitor cocktail, Sigma P8340) was added, and the sample was incubated on ice for 30 min. Samples were then centrifuged for 20 min (16,000×g, 4° C.), and the filtrate was collected and stored at −80° C. Blood samples were collected into EDTA-coated collection tubes. Samples were centrifuged at 2000×g for 5 min to collect plasma, which was stored at −80° C. until use. Cytokine content of vaginal fluid and plasma samples was analyzed with the Cytokine 29-Plex Monkey Panel (Thermo Fisher Scientific) using a Luminex 200 instrument (Luminex Corporation). For cytokines with at least two uncensored data points, censored data below the detection limit was imputed using maximum likelihood estimation assuming a log normal distribution. Cytokine concentrations before and after nanoparticle delivery were compared by t-test using the mean and standard deviation of 1000 imputations.

On day 7, animals were euthanized 4 hrs after final delivery, and collected tissue was fixed in 10% neutral buffered formalin for 7 days. Fixed samples were embedded in paraffin, cut into 5 μm sections, and stained with Hematoxylin and eosin (H&E). Tissue sections were then examined by a pathologist for signs of abnormal features. Results and Discussion

### Nanoparticle Fabrication and Characterization

PLA-HPG NNPs were prepared by a single emulsion method and converted to BNPs by oxidation of vicinal diols on the HPG surface. For PET imaging, ⁸⁹Zr was bound to DFO-BNPs conjugates. The hydrodynamic diameter of NNPs, BNPs, and DFO-BNPs ranged from 98-143 nm (Table 1). The zeta potential of NNPs and BNPs was negative (−43 to −24 mV), but a positive zeta potential (27 mV) was observed after DFO conjugation. The positive surface charge is likely due to the presence of amine groups in the DFO molecule.

TABLE 1
PLA-HPG NNP, BNP, and DFO-BNP characterization.

| | | | | Zeta Potential |
|---|---|---|---|---|
| Nanoparticle | n | Size (nm) | PDI | (mV) |
| Blank NNP | 2 | 114 ± 2.9 | 0.16 ± 0.00 | −24.4 ± 3.4 |
| FITC-NNP | 18 | 122 ± 2.3 | 0.17 ± 0.01 | −28.9 ± 1.7 |
| Blank BNP | 3 | 97.8 ± 1.1 | 0.19 ± 0.02 | −27.2 ± 4.9 |
| FITC-BNP | 4 | 143 ± 8.9 | 0.19 ± 0.01 | −43.4 ± 1.4 |
| DFO-BNP | 3 | 97.6 ± 2.2 | 0.16 ± 0.02 | 27.1 ± 2.3 |

### Characterization of ⁸⁹Zr-BNPs

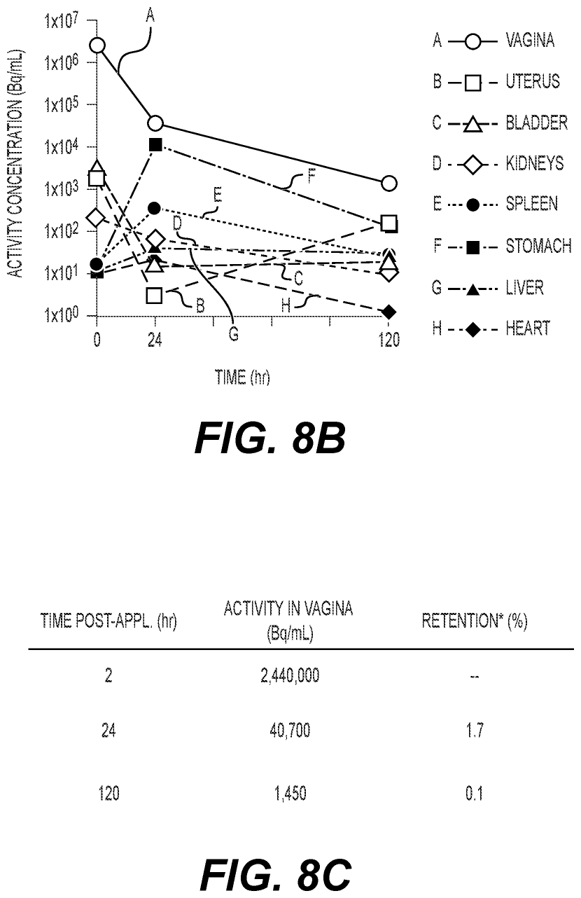

The radiochemical yield of the ⁸⁹Zr-BNPs was found to be 99.3% as determined by radio-TLC. As a control, ⁸⁹Zr-DFO was synthesized with a radiochemical yield of 99.6%. Characterization of ⁸⁹Zr-BNP stability in vitro indicates that ˜30% of ⁸⁹Zr is released from the BNPs over 6 days (FIG. 8A). However, the in vivo studies suggest the chelation is stable: signal from ⁸⁹Zr-BNPs was retained in the reproductive tract, whereas signal from ⁸⁹Zr-DFO was found in multiple organs and in the blood after just 2 hours (see FIGS. 8B-8C and Table 2). This shows that—since [⁸⁹Zr]Zr-DFO can be absorbed through the vaginal epithelium—⁸⁹Zr remains bound to BNPs over the timescale of the experiment. One explanation for the discrepancy between in vitro and in vivo stability is that ⁸⁹Zr is released from BNPs so slowly in vivo that systemic uptake of released ⁸⁹Zr was too low to detect. It is also possible that the in vitro experimental design artificially increased release of ⁸⁹Zr through the multiple wash/centrifugation steps. Further investigation is needed to fully characterize the stability of 89Zr-BNPs.

### ⁸⁹Zr-BNP Retention and Distribution

To confirm that BNPs are not absorbed into systemic circulation after vaginal administration—and that ⁸⁹Zr remains bound to BNPs—vaginal administration of ⁸⁹Zr-BNPs was compared to a smaller molecule, ⁸⁹Zr-DFO (650 Da). To minimize partial volume effects (PVE) in the bladder and uterus, ROI in these regions were manually eroded to ensure separation from the vaginal ROI. For 2 hrs after vaginal application, ⁸⁹Zr-BNP concentrations remained steady in the vagina and did not increase in most organs. In contrast, ⁸⁹Zr-DFO concentration in the kidney increased starting around 60 min after vaginal administration. By 120 min, kidney activity increased 6.4-fold over initial values, showing that ⁸⁹Zr-DFO was taken up systemically from the vagina and filtered in the kidneys. These findings were further confirmed by measuring radioactivity in blood samples taken directly after the 2-hr scans. Blood activity levels in the ⁸⁹Zr-BNP-treated animal were negligible (1.3 Bq/mL at 2 hrs) compared to the ⁸⁹Zr-DFO-treated animal (994 Bq/mL at 2 hrs, Table 2). Overall, these findings show that ⁸⁹Zr remains stably bound to BNPs after in vivo administration. They also indicate that BNPs do not translocate from the vagina into the uterus or systemic circulation, which is an important consideration for safe topical administration.

To quantify vaginal retention, ⁸⁹Zr-BNPs were administered topically to the vaginal canal, and whole-body PET images were taken immediately after administration and after 24 and 120 hrs. Any leaked ⁸⁹Zr-BNPs during administration and post-procedure transport were collected in a diaper, which was measured with an additional PET scan to quantify the residual 89Zr-BNP. It is estimated that 57% of ⁸⁹Zr-BNP radioactivity was immediately lost to leakage during administration and post-scan transport on Day 0. The activity concentration in the vaginal canal after administration was 2.44×106 Bq/mL, which decreased to 40,700 Bq/mL over 24 hrs—a retention rate of 1.7% (FIGS. 8B-8C). After 5 days, ⁸⁹Zr-BNPs were still detected in the vaginal canal at a concentration of 1,450 Bq/mL, which is 0.1% of the administered dose. In a similar study measuring the retention of ⁹⁹mTc-labeled vaginal gels and creams in humans, Chatterton et al. reported an average retention of 40% over 24 hrs for both dosage forms. However, Chatterton et al. administered 0.5 mL of gel or cream to women, whereas the same volume was administered to NHPs with an average weight of 6.1 kg. The significant leakage observed during application and transport shows that the volume administered exceeded the amount needed to coat vaginal epithelium in cynomolgus monkeys. It is speculated that delivering a smaller volume could improve the quality and consistency of vaginal retention estimates.

After administration, ⁸⁹Zr-BNP were primarily located in the upper vagina and around the vaginal opening surrounding skin, likely due to leakage of excess dosage volume. On subsequent scans, signal within the vagina and substantial activity on the external genitalia and surrounding skin were observed. Retention on skin demonstrated that BNPs can effectively bind to the stratum corneum for several days. The binding of BNPs to the external genitalia is an important factor to consider for translation to human use.

In addition to vaginal uptake of ⁸⁹Zr-BNP, off-target uptake in the stomach and digestive tract was observed during the 1- and 5-day scans. It is hypothesized that this uptake is from ingestion of ⁸⁹Zr-BNPs that leaked from the vaginal canal onto food that was consumed by the animal. This is further supported by the signal observed on the animal's hand during the 24-hr scan. Apart from the vaginal canal, the next two regions with the highest radioactivity during Day 0 scan were the bladder and uterus, though the signal was just 0.10% and 0.08% of the vaginal activity, respectively. This is likely attributed to partial volume effects (spill-out from the vagina), rather than translocation of 89Zr-BNPs, despite efforts to minimize these effects by using smaller ROI masks for the bladder and uterus.

TABLE 2
Decay-corrected activity measured in blood samples
during ⁸⁹Zr-BNP and ⁸⁹Zr-DFO studies.

| | Time Post- | Blood Activity |
|---|---|---|
| | Application | Concentration |
| Radiotracer | (hr) | (Bq/mL) |
| ⁸⁹Zr-BNP | 2 | 1.3 |
| ⁸⁹Zr-BNP | 24 | 8.7 |
| ⁸⁹Zr-BNP | 120 | 3.6 |
| ⁸⁹Zr-DFO | 2 | 994 |

### Vaginal Safety

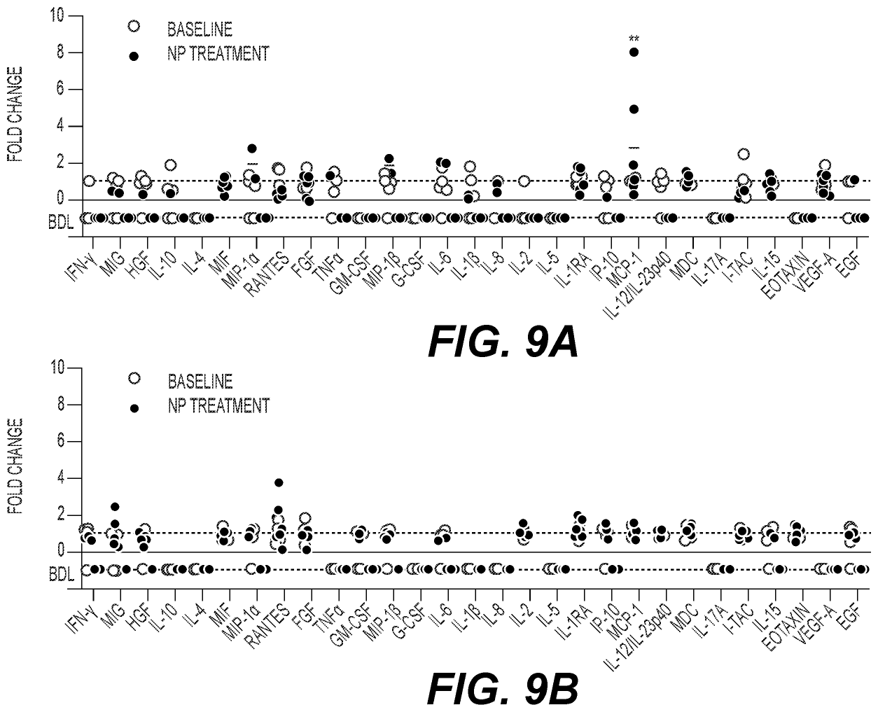

The safety of BNPs for vaginal use was evaluated by monitoring inflammatory biomarker levels in vaginal fluid and plasma before and after administration of multiple nanoparticle doses. There were no significant differences in most vaginal fluid cytokine levels, including important vaginal safety biomarkers such as TNFα, IL-6, MIP-1α, IL-8, IL-1RA, and IL-1β (FIG. 9A). Several key cytokines, such as IFN-γ and GM-CSF were below the detection limit for baseline and post-treatment samples. A slight elevation in MCP-1 in two of the vaginal fluid samples was observed. Although this could be an indication of an inflammatory response to BNPs, one would expect to see increased expression of other cytokines. MCP-1 has also been demonstrated to play an important role in injury and wound healing. Minor vaginal injury, potentially caused by dose administration or vaginal fluid sampling, could be a potential explanation for elevated MCP-1 levels. To test for systemic reaction to BNP administration, biomarker levels in plasma were monitored (FIG. 9B). There were no significant increases in expression for all cytokines tested.

Urogenital tissue samples were collected at the end of the BNP delivery regimen and examined by a pathologist. No abnormal pathologic features were observed in any samples, and there was no evidence of neutrophilic or lymphocytic inflammation. Taken together, the results of the biomarker assessment and histopathological survey show that BNPs are non-toxic and safe for vaginal use.

### Pilot Study

A smaller volume of nanoparticles may be administered to improve the accuracy of retention calculations. A volume of 0.5 mL was applied in the pilot study, but a scan of the diaper used to contain leakage during post-scan transport suggests that 57% of the dose was lost immediately. The optimal dose volume would coat the vaginal epithelium without significant leakage. Alternatively or additionally, nanoparticles could be formulated in a gel or suppository to improve retention. Further, imaging analysis may be improved by development of automatic ROI segmentation approaches for non-human primate CT scans, which would ensure consistency and objectivity between scans. Finally, there was significant signal spill-out from the vagina into the surrounding organs (uterus and bladder). To accurately quantify activity levels, ROI boundaries need to be adjusted to exclude the region adjacent to the vaginal canal.

### CONCLUSION

The female reproductive tract is the entry site for numerous viruses and bacteria that cause infections ranging from HIV to bacterial vaginosis. There is a long-standing interest in developing topical therapeutic formulations for treatment of vaginal infections, but topical drug application to the vagina poses many challenges, including the mucus barrier and an acidic microenvironment from lactic acid-excreting lactobacillus colonizing the vaginal canal. Standard formulations for topical delivery to the vagina include creams, gels, tablets, suppositories, and vaginal rings. There is also growing interest in the use of nanoparticles to protect small molecules from mucus enzymes and the low pH environment—and to provide long-term retention by penetrating through or adhering to the mucus barrier. For example, many researchers have demonstrated that coating nanoparticles in a hydrophilic, neutrally-charged polymer like polyethylene glycol (PEG) or poloxamers can significantly improve nanoparticle diffusion through mucus.

This study demonstrates that a bioadhesive nanoparticle (BNP) formulation that is retained in the vaginal canal for at least 72 hours in mice, significantly outperforming the non-adhesive, mucus-penetrating analogue. The nanoparticles are formed with a block copolymer of polylactic acid (PLA) and hyperbranched polyglycerol (HPG). Under oxidative conditions, vicinal diols on the surface of the nanoparticles can be converted to aldehydes, which bond to protein amine groups through Schiff base interactions. It was demonstrated in mice that these BNPs are non-toxic and can be detected in vaginal epithelial cells and leukocytes for up to 72 hours post administration. This study confirmed the results in non-human primates (NHPs). BNPs were retained in the vaginal canal for at least 5 days and did not enter the uterus or systemic circulation. By contrast, a small molecule (⁸⁹Zr-DFO), was detected in systemic circulation just 60 min after vaginal administration. Multiple vaginal doses of BNPs did not increase the expression of inflammatory biomarkers in vaginal fluid or plasma, suggesting that the nanoparticles are biocompatible. These findings support the further development of this nanoparticle carrier into potential therapeutic applications.

Using PET imaging with ⁸⁹Zr-labeled BNPs, BNPs were detected in the vaginal canal for 5 days. The results indicate that the nanoparticles do not translocate to the uterus or into systemic circulation. Analysis of inflammatory biomarkers in the vaginal fluid and plasma indicates that BNPs are safe and biocompatible, even after multiple doses. BNPs are a promising delivery vehicle for vaginally administered therapeutics, and further research in non-human primates and humans is warranted.

Modifications and variations of the compositions and use thereof will be obvious to those skilled in the art and are intended to come within the scope of the appended claims.

1. An intravaginal insert comprising two or more compartments releasing therapeutic, prophylactic or diagnostic agent at two different times and/or at two different rates of release.

2. The intravaginal insert of claim 1 wherein the compartments contain nanoparticles having a diameter between 40 nm and 500 nm comprising therapeutic, prophylactic or diagnostic agent to be delivered.

3. The intravaginal insert of claim 1 wherein the nanoparticles comprise surface hydroxyl groups effective to promote mucosal penetration.

4. The intravaginal insert of claim 1 wherein the nanoparticles comprise a hydrophobic polymeric core conjugated via a hydrolyzable linker to a branched, star or dendrimeric polyoxyethylene polymer or hyperbranched polyglycerol polymer.

5. The intravaginal insert of claim 4 wherein the hydrophobic polymer is selected from the group consisting of polyhydroxy esters, polyanhydrides, polyorthoesters, and polyhydroxyalkanoates.

6. The intravaginal insert of claim 4 wherein hydroxyl groups on the surface of the branched, star or dendrimeric polyoxyethylene polymer or hyperbranched polyglycerol polymer are released from the polymer in the presence of low pH less than 5.

7. The intravaginal insert of claim 6 wherein nanoparticles are formed of hydrophobic polymer comprising functional adhesive groups exposed when the surface hydroxyl groups are removed.

8. The intravaginal insert of claim 7 wherein the groups are aldehydes or amines.

9. The intravaginal insert of claim 1 wherein the agent is selected from the group consisting of proteins and peptides, nucleic acids, sugars and polysaccharides, small molecules (typically under 1500 Daltons), lipids and lipoproteins

10. The intravaginal insert of claim 1 comprising therapeutic or prophylactic agent selected from the group consisting of hormones, antifungal, antibiotic and antiviral agents.

11. The intravaginal insert of claim 1 comprising two or more compartments sealed at one end with a non-degradable polymer and at the other end with a biodegradable polymer, wherein the biodegradable polymer is selected to degrade at different times and/or with different release kinetics.

12. A method for intravaginal delivery of agent comprising providing the intravaginal insert of claim 1.

13. A method of making the intravaginal insert of claim 1, comprising
forming a non-degradable polymeric intravaginal ring for use in a woman,
creating two or more compartments in the intravaginal ring,
wherein the compartments are closed at one end, either by the polymeric matrix forming the intravaginal ring or by a separate non-degradable polymeric cap.

14. The method of claim 13 further comprising placing in the compartments nanoparticles having a diameter between 40 nm and 500 nm comprising therapeutic, prophylactic or diagnostic agent to be delivered and sealing the compartments with a degradable polymeric cap.

15. The method of claim 14 wherein the nanoparticles comprise surface hydroxyl groups effective to promote mucosal penetration, preferably wherein the nanoparticles comprise a hydrophobic polymeric core conjugated via a hydrolyzable linker to a branched, star or dendrimeric polyoxyethylene polymer or hyperbranched polyglycerol polymer.

16. The method of claim 15 wherein the hydrophobic polymer is selected from the group consisting of polyhydroxy esters, polyanhydrides, polyorthoesters, and polyhydroxyalkanoates, preferably wherein hydroxyl groups on the surface of the branched, star or dendrimeric polyoxyethylene polymer or hyperbranched polyglycerol polymer are released from the polymer in the presence of low pH less than 5.

17. The method of claim 14 wherein the nanoparticles are formed of hydrophobic polymer comprising functional adhesive groups exposed when the surface hydroxyl groups are removed, preferably wherein the groups are aldehydes or amines.

18. The method of claim 14 wherein the nanoparticles comprise agent selected from the group consisting of proteins and peptides, nucleic acids, sugars and polysaccharides, small molecules (typically under 1500 Daltons), lipids and lipoproteins

19. The method of claim 14 wherein the intravaginal insert comprises a therapeutic or prophylactic agent selected from the group consisting of hormones, antifungal, antibiotic and antiviral agents.

20. The method of claim 13 comprising sealing the open end of the compartments with degradable polymer selected to degrade at different times and/or with different release kinetics.
